(19) United States
(12) Patent Application Publication
(10) Pub. No.: US 2024/0276935 A1
(43) Pub. Date: Aug. 22, 2024

(54) METHODS FOR ENHANCING DISEASE RESISTANCE AND INCREASING BIOMASS YIELD AND SECONDARY METABOLITE QUANTITIES IN CANNABIS SATIVA UTILIZING PLANT DEFENSE-RESPONSE ELICITORS

(71) Applicant: Onie Tsabari, Petah Tikva (IL)

(72) Inventor: Onie Tsabari, Petah Tikva (IL)

(21) Appl. No.: 18/437,249

(22) Filed: Feb. 9, 2024

Related U.S. Application Data
(60) Provisional application No. 63/444,572, filed on Feb. 10, 2023.

(51) Int. Cl. A01H 1/00; A01H 6/28; C12N 9/42

(52) U.S. Cl. CPC A01H 1/121; A01H 6/28; C12N 9/244; C12N 9/2442

(57) ABSTRACT

The present invention discloses methods for improving desired qualities in Cannabis sativa L. Methods include the step of: applying a composition having at least one biostimulant, as an elicitor, to Cannabis sativa L., wherein the at least one biostimulant is at least one material selected from the group consisting of: an exogenous polysaccharide, a peptidoglycan, a peptide, a lipopolysaccharide, a modified lipid, and an enzyme, wherein the at least one biostimulant is derived from at least one source material selected from the group consisting of: plants, bacteria, archaebacteria, fungi, animals, protozoa, algae, and viruses, and wherein the at least one biostimulant is known to be sensed by plant surface-receptors to elicit a plant defense-response. Alternatively, the at least one biostimulant is at least one material selected from the group consisting of: a Pathogen-Associated Molecular Pattern (PAMP), a Microbial-Associated Molecular Pattern (MAMP), and an Herbivore-Associated Molecular Patterns (HAMP).

### CROSS REFERENCE TO RELATED APPLICATIONS

This patent application claims priority under 35 U.S.C. §119(e) to U.S. Provisional Patent Application No. 63/444,572, filed Feb. 10, 2023, which is hereby incorporated by reference in its entirety.

### FIELD AND BACKGROUND OF THE INVENTION

The present invention relates to methods for enhancing disease resistance and increasing biomass yield and secondary metabolite quantities in cannabis (i.e., Cannabis sativa L.) utilizing plant defense-response elicitors. In particular, the present invention relates to the fields of agricultural biotechnology, plant growth methodology, and plant development regulation via plant supplements for improving plant productivity and/or harvestable crop value.

Plants produce a wide range of compounds classified as secondary (non-essential) metabolites. Secondary metabolites include a multitude of commercially-important medicinal compounds and other compounds of interest. The secondary metabolite profiles of plants are highly variable among species and cultivars, and are greatly influenced by both biotic and abiotic conditions. Pathogen attack, including bacterial, protozoan, fungal, or insect herbivory, has been shown to induce changes in plant metabolism to counter such attacks. Among others, differential gene expression mediates changes in the plant secondary metabolite profiles.

Such changes are sometimes desired, such as the changes which occur in tea varieties exposed to insect herbivores, marketed as Eastern Beauty Oolong (Dong Fang Mei Ren) with a much higher commercial value. While enhancing desirable qualities by exploiting such changes, using insects to achieve such changes incurs significant losses in yield due to herbivory effects (Kostanda et al., 2022).

Cannabis sativa, a plant known for its medicinal and recreational properties, is susceptible to various fungal pathogens, affecting its yield and quality. Traditional chemical pesticides pose health risks; therefore, there is a need for safer, more effective disease management strategies.

Cannabis produces at least 90 different cannabinoid compounds. The medicinal value of most of these compounds has not been fully assessed. Although CBDA and THCA are the major components of cannabis inflorescence, many others have been shown to exhibit medicinal qualities. Beyond cannabinoids, other types of molecules, such as terpenes, flavonoids, stilbenoids, amino acids, fatty acids, alkaloids, hydrocarbons, carbohydrates, and phenols are produced in cannabis. Many terpenes were shown to exhibit synergistic activity with cannabinoids as well. As such, some cultivars have been shown to be more suitable for certain medical conditions. Indeed, medical cannabis is used for treatment of a myriad of ailments, including multiple sclerosis, epilepsy, neuropathic pain, arthritis, nausea and vomiting due to chemotherapy, appetite stimulation in HIV/AIDS, depression, anxiety disorders, sleep disorders, psychosis, glaucoma, and Tourette Syndrome.

With such a wide array of illnesses and symptoms treated by medical cannabis, it is not surprising that some strains were found to be more effective for specific treatments, such as cannabis strains EP-1 and Avidekel for treating children with autism spectrum disorder (ASD), apparently due to their specific CBDV/CBD ratios. Similarly, specific strains and ratios are suggested to be more effective for treatments of glaucoma and Tourette Syndrome. Such strain-specific effects are largely due to the variability in secondary metabolite profiles of different strains and the complexity of the endocannabinoid system, which modulates the release of most neurotransmitters in the brain including dopamine, glutamate, GABA, opioid peptides, and acetylcholine. As such, different cannabinoids in concert with additional secondary metabolites modulate different receptor systems in the brain and affect neurotransmitter release.

In the prior art, US Patent Publication No. 2015/0230462 A1 by Scheer et al. recites methods for improving germination and stress tolerance characteristics with jasmonates including exposing a seed to a formulation containing methyl dihydrojasmonate (MDHJ) at an amount and duration effective for improving a germination characteristic of the seed. CN U.S. Pat. No. 10,574,6520A teaches application of nano-chitin in improvement of yield and quality of tobaccos.

U.S. Pat. No. 11,638,427 B2 by Key et al. teaches methods and compositions for altering secondary metabolites in plants including applying an effective amount of at least one elicitor, wherein the elicitor is a jasmonate or a salicylate, and combinations thereof. The disclosure further teaches compositions comprising effective amounts of the elicitors disclosed here. The disclosure further relates to methods and compositions for controlling plant pathogens, such as fungal pathogens. U.S. Pat. No. 11,147,271 B2 by Key and US Patent Publication No. 2023/0200385 A1 by Key et al. recites similarly-related subject matter.

It would be desirable to have methods for methods for enhancing disease resistance and increasing biomass yield and secondary metabolite quantities in cannabis utilizing plant defense-response elicitors. Such methods would, inter alia, overcome the various limitations mentioned above.

### SUMMARY OF THE INVENTION

It is the purpose of the present invention to provide methods for enhancing disease resistance and increasing biomass yield and secondary metabolite quantities in cannabis utilizing plant defense-response elicitors.

It is noted that the term “exemplary” is used herein to refer to examples of embodiments and/or implementations, and is not meant to necessarily convey a more-desirable use-case. Similarly, the terms “alternative” and “alternatively” are used herein to refer to an example out of an assortment of contemplated embodiments and/or implementations, and is not meant to necessarily convey a more-desirable use-case. Therefore, it is understood from the above that “exemplary” and “alternative” may be applied herein to multiple embodiments and/or implementations. Various combinations of such alternative and/or exemplary embodiments are also contemplated herein.

Embodiments of the present invention provide methods for enhancing disease resistance and secondary metabolite production in Cannabis sativa. Such methods involve applying specific biostimulants, including plant-, fungal-, or bacterial-derived elicitors, to Cannabis sativa plants. Such methods exploit changes in plant secondary metabolite profiles by utilizing elicitor compounds (e.g., PAMPs (Pathogen-Associated Molecular Patterns) and/or HAMPs (Herbivore-Associated Molecular Patterns)) to induce plant defense responses.

Application of such elicitors to induce plant defense is expected to alter the secondary metabolite profile in various ways. Since enhancing desirable qualities could imply reducing or increasing specific compounds, depending on the medicinal value of various compounds in for a given application, there is significant benefit in methods for altering the secondary metabolite profile of cannabis in various manners. Such modification, whether increasing or decreasing the relative amounts of certain substances, of compound ratios can be tailored to optimize patient treatments for targeted conditions. Concomitantly, such applications are expected to prime plant defense response and enhance their resilience against various pathogens.

Embodiments of the present invention provide methods for altering the production of one or more secondary plant metabolites in cannabis utilizing one or more elicitor, wherein the elicitor is a natural or synthetic material containing PAMPS (also referred to as MAMPS—Microbial-Associated Molecular Patterns), HAMPs, and/or DAMPs (Damage-Associated Molecular Patterns). These compounds, derived from plant pathogens (bacterial, protozoan, fungal, and/or insect herbivores) or mechanical injury to plant tissues, have been shown to alter the secondary metabolite profile of various plant species. Compounds include chitin in conjugated and polymer forms, chitosan, bruchins esters, tatty acid-amino acid conjugates (such as volicitin), sulfooxy fatty acids (such as compounds belonging to the caeliferins), peptides (such as inceptin and alamethicin), cell wall derivatives (such as oligogalacturonides), and/or enzymes (such as glucose oxidases, β-glucosidases).

The compounds can be produced from natural sources or synthetically; applied topically by spray, injection, dissemination, or other application; systemically through watering treatment, or in any way applied to the whole plant or parts of the plant at any stage of the growth cycle, from germination to harvest.

Therefore, according to the present invention, there is provided for the first time a method for improving desired qualities in Cannabis sativa L., the method including the step of: (a) applying a composition having at least one biostimulant, as an elicitor, to Cannabis sativa L., wherein the at least one biostimulant is at least one material selected from the group consisting of: an exogenous polysaccharide, a peptidoglycan, a peptide, a lipopolysaccharide, a modified lipid, and an enzyme, wherein the at least one biostimulant is derived from at least one source material selected from the group consisting of: plants, bacteria, archaebacteria, fungi, animals, protozoa, algae, and viruses, and wherein the at least one biostimulant is known to be sensed by plant surface-receptors to elicit a plant defense-response.

Alternatively, the at least one biostimulant is at least one material selected from the group consisting of: a Pathogen-Associated Molecular Pattern (PAMP), a Microbial-Associated Molecular Pattern (MAMP), and an Herbivore-Associated Molecular Patterns (HAMP), and wherein the PAMP and the HAMP are derived from at least one source material selected from the group consisting of: fungi, bacteria, archaebacteria, lichen, mollusks, insects, arthropods, oomycete, viruses, algae, and protozoa.

Alternatively, the enzyme is a cell-wall-degrading enzyme.

Most alternatively, the cell-wall-degrading enzyme is at least one material selected from the group consisting of: ligninolytic enzymes, hemicellulases, pectinases, and cellulases.

Alternatively, at least one of the at least one biostimulant is a Damage-Associated Molecular Pattern (DAMP), and wherein the DAMP is derived from at least one source material selected from the group consisting of: plants, moss, lichen, algae, enzymes, biopolymers, and degradation products thereof.

More alternatively, the biopolymers are materials selected from the group consisting of: cellulose, hemicellulose, pectin, and degradation products thereof.

More alternatively, the biopolymers are materials selected from the group consisting of: lignin polyphenols and degradation products thereof.

More alternatively, the enzymes are Pathogenesis-Related Proteins (PRs).

Most alternatively, the Pathogenesis-Related Proteins are materials selected from the group consisting of: β-1,3 glucanases and chitinases.

Alternatively, the plant defense-response is an increase in at least one aspect selected from the group consisting of: a disease resistance, a secondary metabolite, and a biomass yield.

Most alternatively, the disease resistance includes resistance to important plant fungal pathogens in all phases of growth for both rooting clones and adult cannabis sativa plants.

Most alternatively, the secondary metabolite is at least one material selected from the group consisting of: a cannabinoid and a terpenoid, and wherein the increase in the secondary metabolite is in a cultivar-dependent manner.

Alternatively, the step of applying the composition is performed by using at least one technique selected from the group consisting of: employing a foliar spray and employing substrate-drenching.

Alternatively, the at least one biostimulant is in at least one form selected from the group consisting of: a water-soluble material, a crystalline material, an amorphous material, a non-soluble material, a suspended colloidal particle, a nanoparticle constituent, a phase-boundary aggregate, a micellar suspension, and a precipitate.

Alternatively, the exogenous polysaccharide includes at least one material selected from the group consisting of: a polysaccharide conjugate, a polysaccharide derivative, a surface-modified polysaccharide, a depolymerized polysaccharide, and an oligomer-fragmented polysaccharide.

These and further embodiments will be apparent from the detailed description and examples that follow.

### BRIEF DESCRIPTION OF THE DRAWINGS

The present invention is herein described, by way of example only, with reference to the accompanying drawings, wherein:

FIG. 1A depicts a graph showing the protective effect of PAMP polysaccharides and enzymes on mature plants of C. sativa against the mold pathogen of Butyris cinerea, according to embodiments of the present invention;

FIG. 1B depicts a graph showing the protective effects of PAMP polysaccharides and enzymes on mature plants of C. sativa against the mold pathogen of Fusarium sp., according to embodiments of the present invention;

FIG. 1C depicts a graph showing the protective effects of PAMP polysaccharides and enzymes on mature plants of C. sativa against the mold pathogen of Golovinomyces sp., according to embodiments of the present invention.

### DESCRIPTION OF THE ILLUSTRATIVE EMBODIMENTS

The present invention relates to methods for enhancing disease resistance and increasing biomass yield and secondary metabolite quantities in cannabis utilizing plant defense-response elicitors. The principles and operation for providing such methods, according to the present invention, may be better understood with reference to the accompanying description and drawings.

The most destructive root pathogens of cannabis are Fusarium and Pythium species particularly when infections occur during the rooting phase or vegetative growth. symptoms include yellowing of foliage and stem necrosis, and the related disease pathology is typically vascular wilt, but several Fusarium species can also result in seedling damping-off, crown rot, and reduced growth of stems and roots. These established infections may progress into the flowering stage, causing stunting and ultimately plant death. If Fusarium and Pythium occur concurrently on root and crown tissues, severe symptoms, such as sudden and rapid death of flowering plants, can occur. Losses caused by these two pathogens can be as high as 30%, leading to substantial economic losses.

Cannabis is also susceptible to the gray mold disease caused by Botrytis cinerea. This air-borne necrotrophic pathogen can attack cannabis seeds, leaves, inflorescences, and stalks, causing lesions covered by a conidia grey layer and often leading to broader crop decay.

Additionally, certain strains of cannabis are vulnerable to powdery mildew (PM) disease caused by Golovinomyces spp., which can reduce its yield and photosynthesis rate by damaging foliage and preventing the light from reaching its surface, resulting in premature plant senescence. PM represents a relevant limitation for cannabis production, adversely affecting both harvest yield and quality. (see Sirangelo et al. and Punja in Literature section)

Pathogens impact regulatory requirements. Dried cannabis products must have minimal contamination of the inflorescences (buds) by fungi, yeasts, and bacteria, as well as by specific coliform bacteria, chemical pesticides and mycotoxins. Products failing to meet the minimum threshold requirement for these contaminants cannot be sold. Limits imposed for culturable colony-forming units mold vary depending on specific countries, and can range from <1,000 to >10,000 cfu/g.

The use of pesticides against fungal pathogens in cannabis could have health risks for the consumer, and alternative methods include environmental control and applications of rhizobacteria promoting plant growth. Currently, several products to manage PM in cannabis are available, like the bio-fungicide Regalia Maxx (an extract of giant knotweed). (see Jamiołkowska, Stasinska-Jakuba et al., and Rouphael et al. in Literature section). Elicitor compounds are considered plant biostimulants.

“Plant biostimulants are substances and materials, with the exception of nutrients and pesticides, which, when applied to plant, seeds or growing substrates in specific formulations, have the capacity to modify physiological processes of plants in a way that provides potential benefits to growth, development and/or stress responses.” du Jardin (2012) (see Rouphael et al. therein in Literature section)

Generally speaking, a “plant biostimulant” is a naturally-occurring substance or microbe that is used either by itself or in combination with other naturally-occurring substances or microbes for the purpose of stimulating natural processes in plants or in the soil in order to, among other things, improve nutrient and/or water use efficiency by plants, help plants tolerate abiotic stress, or improve the physical, chemical, and/or biological characteristics of the soil as a medium for plant growth. (see Draft Guidance for Plant Regulator Label Claims, Including Plant Biostimulant, United States Environmental Protection Agency (US-EPA))

These elicitor compounds or biostimulants activate plant defense responses against pathogens. To fight diseases, plants activate a complex network of defense pathways, which allow them to respond to the pathogen. Plant cells recognize pathogens rapidly and activate the production of pathogenesis-related proteins (PRs), and increase the production of hormones such as SA, JA, ET, abscisic acid (ABA), and brassinosteroids (BR), which have been known to play a key role in defense against this pathogen. ET and JA have synergistic effects in plant B. cinerea resistance. JA targets the JA-Zim (JAZ) repressor for degradation, activates JA/ET-related defense genes, and SA negatively regulates this transcriptional cascade. (see Sirangelo et al. in Literature section)

Elaborating on the recognition pathways, plants solely depend upon the intracellular or cell surface receptors during pathogen attack (Zipfel, 2014). Either the degradation products of the host cell wall components or the pathogen and its associated molecules initiate the immune responses which include peptides, lipopolysaccharides, chitin, chitosan, and oligosaccharides respectively. These alarm signatures are described as elicitors and designated as microbe/pathogen-associated molecular patterns (MAMP/PAMPs), and danger/damage-associated molecular patterns (DAMPs) (Anil et al., 2014; Wiesel et al., 2014). The specific pattern-recognition receptors (PRRs) situated on the host cell membrane are involved in the mediation of recognition (Zipfel, 2014) which turns on the signaling cascade triggering the physiological responses in the cell. This first response of the plant immunity system is referred to as PAMP-triggered immunity (PTI).

Effector triggered immune (ETI) responses are discussed in Hopke et al., 2018. ETI involves the recognition of the effector molecule by the host and subsequent mounting of stronger response (an intensified PTI) and is often associated with localized hypersensitive response (HR), a cell death event, and long lasting systemic acquired resistance to protect the unaffected remaining part of the tissues to further attack (Cui et al., 2015). These PAMPs/MAMPs or DAMPs are the signature molecules that elicit defense responses in plants. They may include polysaccharides, proteins, enzymes, and lipopolysaccharides. (see Kongala et al. in Literature section)

The application of hormones induces cannabinoid production. Several phytohormones regulate plant trichome formation and elicit the synthesis of secondary metabolites in many plant species in both in vitro and in vivo systems. Therefore, exogenously delivered plant signaling molecules have the potential to modify the chemical profiles of medical cannabis. It was found that the foliar application of SA, methyl jasmonate (MeJA), and GABA produces changes in the accumulation of the two major cannabinoids, cannabidiolic acid (CBDA) and Δ9-tetrahydrocannabinolic acid (THCA), in leaves and inflorescences of a medical cannabis variety. MeJA at 0.1 mM increased the CBDA content in inflorescences by 15.6%, while SA and MeJA at 0.1 mM increased CBDA and THCA accumulation in leaves by up to 57.3%. (see Garrido et al. in Literature section)

Phytochemical compounds and antioxidant capacity in cannabis sativa were influenced by various concentrations of salicylic acid (SA). The highest TPC, TFC, TCC, Chl a, Chl b, and antioxidant capacity were obtained in 1 M treatment; whereas, the lowest of them were found in control plants. The major cannabinoids in the analyzed extracts were CBD (19.91%-37.81%), followed by A9-THC (10.04%-22.84%), and CBL (nd-14.78%). The highest CBD (37.81%) and A9-THC (22.84%) were obtained in 1 M of SA. (see Mirzamohammad et al. in Literature section)

There is significant medicinal relevance of secondary metabolite profile. Today, patients are commonly treated by specific ‘strains’ of cannabis in the absence of any clear definition of the biological activity or chemical composition of a strain. Based on the major cannabinoid concentrations, five different chemotypes of cannabis are recognized. Drug-type plants that have a high THCA/CBDA ratio (>>1.0) are classified as chemotype I; plants that exhibit an intermediate ratio (usually 0.5-2.0) are classified as chemotype II; typical fiber-type plants that have a low THCA/CBDA ratio (<<1.0) are classified as chemotype III. (see Aizpurua-Olaizola et al. and Jin et al. in Literature section).

However, Cannabis produces a multitude of phytomolecules, including up to 150 different phytocannabinoids and hundreds of other compounds including terpenes and flavonoids, mainly concentrated in the plant inflorescence. In each cannabis strain or ‘chemovar’, tens of phytomolecules are found in particular combinations that confer a superior medical activity versus a single molecule, termed an entourage effect. Recent studies have shown that crude cannabis inflorescence extract exhibits superior activity than single phytomolecules in medical treatments.

The secondary metabolite profile is chemovar dependent. The two major cannabinoids and those best known for their therapeutic potentials are THC and CBD, i.e., the neutral homologues of THCA and CBDA, respectively. THC is the main psychoactive agent of cannabis and has anti-inflammatory analgesic, appetite-stimulant, and antiemetic properties. In contrast, CBD can modulate the euphoric effects of THC and has antipsychotic, neuroprotective, anticancer, antidiabetic, and other positive effects, such as the ability to reduce tobacco addiction.

While much attention has been given to the modulatory actions of A9-THC and CBD, a number of structurally unusual phytocannabinoids with cannabimimetic-like actions have been reported in the past few years, and exploitation of this chemical diversity may be a promising area for ECS drug development (Chianese et al., 2020; Citti et al., 2019a; Pollastro et al., 2011). While these compounds are usually found at levels that are several orders of magnitude lower than CBD and A9-THC. (see Welling et al. in Literature section), medicinal interest in these unusual minor phytochemical analogues is growing rapidly due to their favorable safety profiles and efficacy in preclinical trials (Huizenga et al., 2019; Jadoon et al., 2016).

For example, cannabigerol (CBG) and cannabichromene (CBC) seem to be promising compounds for different medical applications. Although they have not been extensively studied, CBG has promising potential for the treatment of glaucoma, inflammatory bowel disease, and prostate carcinoma. CBC has analgesic effects, the potential to stimulate the growth of brain cells, and the ability to normalize gastrointestinal hypermotility. Elaborating on the importance and variation of the secondary metabolites of cannabinoids and terpenes, terpenes are responsible for the plant's aroma. In addition, they possess specific medical effects and may act synergistically with cannabinoids. In fact, there are several promising applications based on the combined use of cannabinoids and terpenes, such as new acne therapies utilizing CBD with the monoterpenes of limonene, linalool, and pinene; new antiseptic agents with CBG and pinene; treatment of social anxiety disorder using CBD with limonene and linalool; and treatment of sleeping disorders by adding caryophyllene, linalool, and myrcene to 1:1 CBD/THC extracts.

### Experimental Studies

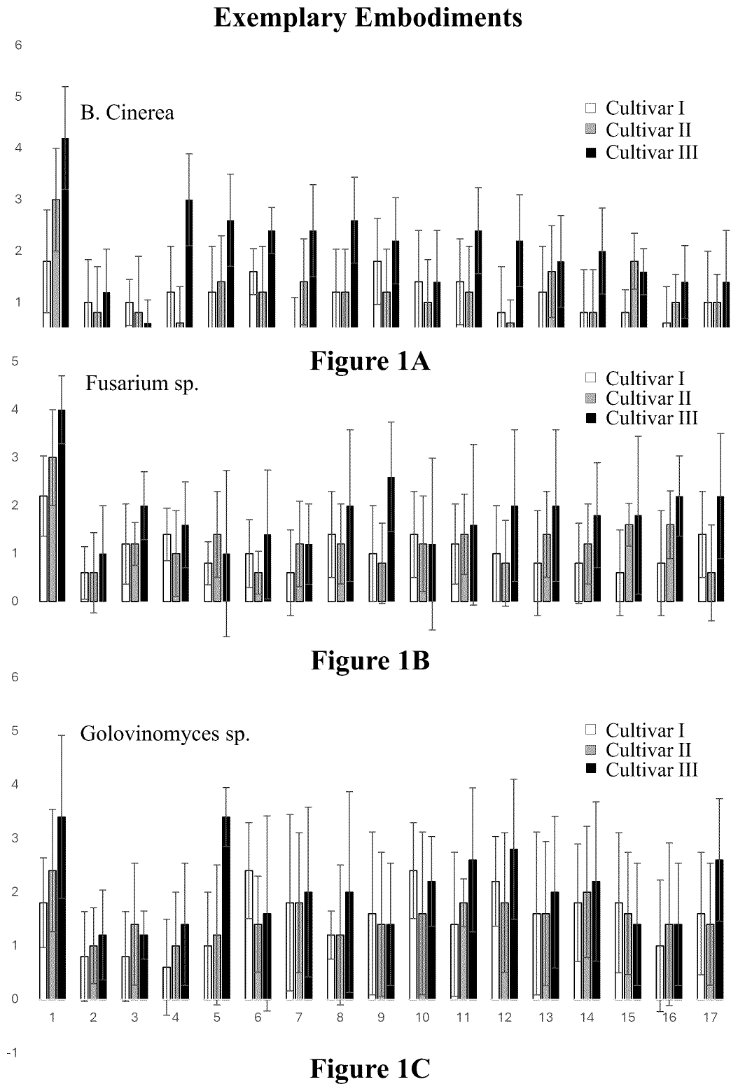

Referring to the drawings, FIG. 1A depicts a graph showing the protective effects of PAMP polysaccharides and enzymes on mature plants of C. sativa against the mold pathogen of Butyris cinerea, according to embodiments of the present invention.

Plants of three cultivars, two high THC strains (cultivars I and II), and one high CBD strain (cultivar III; see also Aizpurua-Olaizola et. al, 2016) were treated with bacterial/fungal PAMP elicitor compounds formulated in water and applied by foliar spray three times per week throughout the vegetative growth phase (i.e., three weeks), and one week into the flowering phase. On the second week of flowering, 15 replicate plants of each treatment per cultivar were taken to a separate chamber to assess mold resistance.

The PAMP elicitor compounds studied (and indicated on the x-axis below FIG. 1C by reference numerals were: a Control-Water (1), Chitin (2), Chitosan (3), N-Acetyl-D-Glucosamine (4), Polygalacturonic Acid (5), Xyloglucans (6), Laminarin (7), N-Acetylmuramic Acid (8), Hyaluronic acid (9), Levan (10), Xanthan (11), Alginate (12), Laminarin (13), Succinoglycan (Rhezoan) (14), Beta Glucosidase (15), Glucose Oxidase Powder (16), and a Mix* (17)—a mixture of chitosan oligomers, hardwood kraton lignin (S-lignin), and beta glucosidase (103 u/ml; extracted from the fungus Trichoderma reesei) suspended in water.

For each cultivar, five plants were inoculated by applying 50ul drop of culture medium containing the fungal pathogen Butyris cinerea ˜103-104 Cfu/ml on two of the bottom fan leaves. Progression of the mold growth was assessed after one week by scoring the visible symptoms into six categories (0 =no growth, 5 =visible sporangium spreading to stem tissue and necrosis of infected leaves).

FIG. 1B depicts a graph showing the protective effects of PAMP polysaccharides and enzymes on mature plants of C. sativa against the mold pathogen of Fusarium sp., according to embodiments of the present invention. Identical experimental procedures to those described with regard to FIG. 1A were conducted in studies involving the pathogen Fusarium sp. For each cultivar, five plants were inoculated by applying 50ul drop of culture medium containing the fungal pathogen Fusarium sp. ˜10³-10⁴ Cfu/ml on two of the bottom fan leaves.

FIG. 1C depicts a graph showing the protective effects of PAMP polysaccharides and enzymes on mature plants of C. sativa against the mold pathogen of Golovinomyces sp., according to embodiments of the present invention. Identical experimental procedures to those described with regard to FIGS. 1A and 1B were conducted in studies involving the pathogen Golovinomyces sp. For each cultivar, five plants were inoculated by applying 50 μl drop of culture medium containing the fungal pathogen Golovinomyces sp. ˜10³-10³ Cfu/ml on two of the bottom fan leaves.

Table 1 below shows the protective effects of PAMP polysaccharides and enzymes on rooting clones of C. sativa against the important mold pathogen Fusarium sp. Cultivar III was shown from the data in FIG. 1B to be most susceptible to mold pathogens. Thus, 10 clones of cultivar III were selected to be treated with formulations containing bacterial/fungal PAMP elicitor compounds (as listed in Table 1) formulated in water. Treatments were performed by spraying the clones daily with ˜2 ml solution/clone, starting one week after cutting. Plants were inoculated two weeks after cutting (one week after treatment application) by applying 10 ml Fusarium culture (˜10³ Cfu/ml) to the water tray. After one hour, clones were removed and placed in clean water trays. Survival was assessed one week later upon reaching the vegetative growth phase. The experiment was repeated three times for a total of 30 replicate clones per elicitor treatment.

The effects on secondary metabolite production, total biomass, and harvest yield were shown to be in agreement with well-documented, published scientific data (elicitors and biostimulants). Several fungal-, plant-, and bacterial-derived exogenous polysaccharides, polyphenols, or enzymes significantly increased total cannabinoid, total terpenoid, and specific secondary metabolite concentrations, and biomass, and harvest yield (dried inflorescence material per plant) in specific cultivars following weekly applications.

TABLE 1
Protective effect of PAMP polysaccharides and
enzymes on rooting clones of C. sativa against
important mold pathogen Fusarium sp.

| Treatment | Mean | | Total | | |
|---|---|---|---|---|---|
| compound | Survival | SD | Alive | Dead | Sig. |
| Control - Water (1) | 36.7% | 11.5% | 12 | 18 | — |
| Chitin (2) | 76.7% | 15.3% | 23 | 7 | P < 0.01 |
| Chitosan (3) | 76.7% | 5.8% | 23 | 7 | P < 0.05 |
| N-Acetyl-D- | 70.0% | 10.0% | 21 | 9 | P < 0.05 |
| Glucosamine (4) | | | | | |
| Polygalacturonic | 70.0% | 10.0% | 21 | 9 | P < 0.05 |
| Acid (5) | | | | | |
| Xyloglucans (6) | 70.0% | 10.0% | 21 | 9 | P < 0.05 |
| Laminarin (7) | 63.3% | 15.3% | 19 | 11 | P < 0.05 |
| N-Acetylmuramic | 70.0% | 10.0% | 21 | 9 | NS |
| Acid (8) | | | | | |
| Hyaluronic acid (9) | 40.0% | 10.0% | 12 | 18 | NS |
| Levan (10) | 46.7% | 15.3% | 14 | 16 | NS |
| Xanthan (11) | 46.7% | 11.5% | 14 | 16 | NS |
| Alginate (12) | 50.0% | 10.0% | 15 | 15 | NS |
| Laminarin (13) | 43.3% | 15.3% | 13 | 17 | NS |
| Succinoglycan | 46.7% | 11.5% | 14 | 16 | NS |
| (Rhezoan) (14) | | | | | |
| Beta Glucosidase (15) | 70.0% | 17.3% | 21 | 9 | P < 0.05 |
| Glucose Oxidase | 70.0% | 17.3% | 21 | 9 | P < 0.05 |
| Powder (16) | | | | | |
| Mix* (17) | 76.7% | 5.8% | 23 | 6 | P < 0.01 |

Table 2 below shows the effects of routine PAMP and DAMP foliar spray applications on dry inflorescence weight. Plants of three cultivars, two high THC strains (cultivars I and II), and one high CBD strain (cultivar III; see also Aizpurua-Olaizola et. al, 2016) were treated with elicitor compounds formulated in water and applied by foliar spray three times per week throughout the vegetative growth phase (i.e., three weeks), and one week into the flowering phase (i.e., seven weeks). At the end of the flowering phase, plants were harvested, dried, and inflorescence parts were separated from the rest of the plant biomass and their weights were recorded. Results of the significance (sig.) shown are the mean inflorescence dry weight and standard deviation (±SD) of five replicate plants per treatment per cultivar.

TABLE 2
Effects of routine PAMP and DAMP foliar spray applications on dry inflorescence weight.

| | Treatment | Cultivar I | | Cultivar II | | Cultivar III | |
|---|---|---|---|---|---|---|---|
| | Elicitor | Yield | | Yield | | Yield | |
| # | Compound | (g/plant) | Sig. | (g/plant) | Sig. | (g/plant) | Sig. |
| 1 | Control | 8.7 ± 0.9 | | 7.7 ± 1.3 | | 8.2 ± 1 4 | |
| 2 | Chitin | 13.7 ± 1.9 | P < 0.05 | 11.1 ± 1.5 | P < 0.05 | 11.5 ± 1.7 | P < 0.05 |
| 3 | Chitosan | 16 ± 1.4 | P < 0.01 | 13.9 ± 1.7 | P < 0.01 | 14.1 ± 1.2 | P < 0.01 |
| 4 | N-Acetyl-D- | 11.6 ± 1.7 | | 10.7 ± 1.9 | | 9.2 ± 0.7 | |
| | Glucosamine/N- | | | | | | |
| | Acetylglucosamine | | | | | | |
| 5 | Polygalacturonic | 14.5 ± 2.6 | P < 0.05 | 12.8 ± 2 | P < 0.01 | 13.7 ± 0.8 | P < 0.01 |
| | Acid | | | | | | |
| 6 | Xyloglucans | 14.4 ± 1.5 | P < 0.05 | 9.3 ± 1.7 | | 10.5 ± 1.7 | |
| 7 | Laminarin | 17.4 ± 1.7 | P < 0.01 | 11.7 ± 1.5 | P < 0.05 | 12.1 ± 1.5 | P < 0.05 |
| 8 | N-Acetylmuramic | 13.4 ± 2.1 | P < 0.05 | 11.5 ± 2 | P < 0.05 | 11.5 ± 1.6 | P < 0.05 |
| | Acid | | | | | | |
| 9 | Hyaluronic Acid | 12.3 ± 1.8 | | 10.3 ± 1.8 | | 10.6 ± 1.6 | |
| 10 | Levan | 12.3 ± 1.7 | | 9.8 ± 1.1 | | 9.3 ± 1 4 | |
| 11 | Xanthan | 14.9 ± 3.1 | P < 0.05 | 11.3 ± 1.5 | P < 0.05 | 11.1 ± 1.5 | |
| 12 | Alginate | 15 ± 2.7 | P < 0.01 | 11.5 ± 2.2 | P < 0.05 | 11 ± 2.3 | |
| 13 | Gellan | 14.2 ± 2.7 | P < 0.05 | 10.3 ± 0.5 | | 10.9 ± 1.9 | |
| 14 | Succinoglycan | 12.2 ± 1.4 | | 11.6 ± 1.6 | P < 0.05 | 10.5 ± 1.5 | |
| | (Rhezoan) | | | | | | |
| 15 | Beta Glucosidase | 12.6 ± 2.1 | P < 0.05 | 11.7 ± 2.2 | P < 0.05 | 12.5 ± 1.7 | P < 0.05 |
| 16 | Glucose Oxidase | 14 ± 2.2 | | 13.1 ± 2.5 | P < 0.01 | 11 ± 1.8 | |
| | Powder | | | | | | |
| 18 | Coniferyl Alcohol | 13.6 ± 2.9 | P < 0.05 | 12.8 ± 1.3 | P < 0.01 | 12.4 ± 1.8 | P < 0.05 |
| 19 | D Galactoronic Acid | 13.5 ± 1.8 | P < 0.05 | 11.5 ± 2.1 | P < 0.05 | 10.2 ± 1.8 | |
| 20 | Hemicellulose | 8.4 ± 1.1 | | 7.5 ± 1.6 | | 7.3 ± 0.9 | |
| 21 | Lichenin | 14.3 ± 1.8 | P < 0.05 | 11.9 ± 1.8 | P < 0.05 | 12.8 ± 1.8 | P < 0.05 |
| 22 | Lignin | 8.8 ± 0.9 | | 8.3 ± 1.2 | | 8.5 ± 1 | |
| 23 | Mannans | 11.2 ± 1.7 | | 9.4 ± 1.3 | | 9.1 ± 0.8 | |
| 24 | Paracoumaryl | 14.7 ± 2.5 | P < 0.05 | 9.2 ± 1 | | 11.9 ± 2.9 | P < 0.05 |
| | Alcohol | | | | | | |
| 25 | Pectin | 8 ± 1 | | 8.8 ± 1.5 | | 8.8 ± 1.7 | |
| 26 | Sinapyl Alcohol | 16.4 ± 1.2 | P < 0.01 | 12.9 ± 2.1 | P < 0.01 | 13.5 ± 2.6 | P < 0.01 |
| | (Syringil monomer) | | | | | | |
| 27 | Xylans | 9.7 ± 1.4 | | 8.8 ± 1.2 | | 8.3 ± 1.9 | |

Tables 3A and 3B below show the effects of routine PAMP and DAMP foliar spray applications on secondary metabolites. Secondary metabolites (total SM) were extracted from three out of the five plants using methanol extraction at room temperature, followed by cannabinoid identification and quantification by HPLC-UV, and terpenoid identification and quantification using GC-MS and GC-FID as previously described (Aizpurua-Olaizola et. al, 2016).

TABLE 3A
Effects of routine PAMPs and DAMP foliar spray applications on secondary metabolites.

| | Treatment elicitor | | | |
|---|---|---|---|---|
| # | compound | Cultivar I | Cultivar II | Cultivar III |
| 2 | Chitin | Limonene, CBDA, | B_curcumene, | B_Eudesmol, |
| | | B_farnasene, | B_farnasene | B_caryophyllene, |
| | | B_curcumene, | | G_Eudesmol, |
| | | A_bisabolol | | B_elemene |
| 3 | Chitosan | Limonene, CBDA, | A_selinene, | B_elemene |
| | | B_farnasene, | CBDA, | |
| | | B_curcumene, Total | Linalool, | |
| | | Terpenes, A_bisabolol, | B_curcumene | |
| | | Ocemene | | |
| 4 | N-Acetyl-D- | Limonene, CBDA, Total | A_selinene, | B_caryophyllene, |
| | Glucosamine/N- | Terpenes, B_farnasene | Linalool | B_elemene |
| | Acetylglucosamine | | | |
| 5 | Polygalacturonic | Limonene, CBDA, | A_bisabolol, | Ocimene, |
| | Acid | B_farnasene, Total | A_selinene, | B_caryophyllene |
| | | Terpenes, A_bisabololo | CBDA | |
| 6 | Xyloglucans | Total Terpenes, CBDA, | A_bisabolol, | B_elemene |
| | | CBC, B_farnasene, | CBDA | |
| | | B_caryophyellen, | | |
| 7 | Laminarin | Total Terpenes | CBDA, | CBDA, B_curcumene, |
| | | THC, CBDA, B_curcume | G_Elemene | B_elemene, |
| | | ne, | | Ocimene |
| 8 | N-Acetylmuramic | Total Terpenes, CBDA, | A_bisabolol, | B_curcumene, |
| | Acid | THC, Limonene, | CBDA | B_elemene |
| 9 | Hyaluronic Acid | Total Terpenes, CBDA, | G_Elemene | THC, |
| | | THC, Limonene, | | B_caryophyllene, |
| | | | | B_curcumene, |
| | | | | B_elemene |
| 10 | Levan | Total Terpenes, CBDA, | G_Elemene, | THC, B_elemene |
| | | THC, B_curcumene, | B_curcumene | |
| | | Limonene, | | |
| 11 | Xanthan | Total Terpenes, CBDA, | | Ocimene, A_bisabolol, |
| | | THC, A_bisabololol | | B_elemene |
| | | B_curcumene, | | |
| 12 | Alginate | Total Terpenes, CBDA, | | Ocimene, |
| | | B_curcumene, | | B_caryophyllene, |
| | | | | B_elemene |
| 13 | Gellan | B_farnasene | | B_curcumene, |
| | | | | B_elemene |
| 14 | Succinoglycan | Total Terpenes, CBDA, | CBDA | Ocimene, THCA, |
| | (Rhezoan) | B_curcumene, | | B_curcumene, |
| | | | | B_elemene |

Statistical comparison of treatment effectiveness was performed by two-way ANOVA. Treatments that induced significant increases (P<0.05) in total SM, total cannabinoids, total terpene, or specific cannabinoids/terpenes are listed in Tables 3A and 3B.

TABLE 3B
Effects of routine PAMPs and DAMP foliar spray
applications on secondary metabolites.

| | Treatment elicitor | | | |
|---|---|---|---|---|
| # | compound | Cultivar I | Cultivar II | Cultivar III |
| 15 | Beta Glucosidase | Total Cannabinoids, | CBDA | Ocimene |
| | | CBDA, CBD Linalool, | | |
| 16 | Glucose Oxidase | Total Cannabinoids, | CBDA | Ocimene, |
| | | Total Terpenes, | | B_curcumene |
| | | Linalool, CBDA, CBD, | | |
| | | CBC B_farnasene, | | |
| 18 | Coniferyl Alcohol | A_bisabolol | Total Terpenes, | Total Cannabinoids, |
| | (G-Lignin) | | A_bisabolol, | Total Terpenes, Total |
| | | | A_pinene, | SM THCA, Ocimene, |
| | | | G_Elemene | A_bisabolol |
| 19 | D Galactoronic | Total Terpenes, | A_pinene, | Total Terpenes, |
| | Acid | Linalool, CBDA, | G_Elemene | Terpenolene, |
| | | B_farnasene, | | A_bisabolol, |
| | | B_caryophyellen | | B_curcumene, |
| 20 | Hemicellulose | Linalool, B_farnasene, | A_pinene | Total Terpenes, |
| | | B_caryophyellene | | Terpenolene, |
| | | | | A_bisabolol, |
| 21 | Lichenin | Total Terpenes, | | |
| | | Linalool, B_farnasene, | | |
| | | B_caryophyellene | | |
| 22 | Lignin | CBDA | | Total Cannabinoids, |
| | | | | Total Terpenes, Total |
| | | | | SM, THCA , THC, |
| | | | | A_bisabolol, Ocimene, |
| 23 | Mannans | CBDA, Linalool, | | A_bisabolol |
| | | B_farnasene, | | |
| | | B_caryophyellene | | |
| 24 | Paracoumaryl | Total Terpenes, CBC | A_pinene, | Total Terpenes, Total |
| | Alcohol | A_bisabololol | G_Elemene | Cannabinoids, Total |
| | (H-Lignin) | | | SM, Ocimene, THC, |
| | | | | CBC A_bisabolol, , |
| | | | | Ocimene, B_elemene |
| 25 | Pectin | CBDA | CBDA | Ocimene |
| 26 | Sinapyl Alcohol | Total SM, Total | Total Terpenes, | Total Terpenes, Total |
| | (S-Lignin) | Terpenes | A_bisabolol, | SM, Total |
| | | | A_pinene, | Cannabinoids, |
| | | | A_selinene, | Ocimene, THCA, |
| | | | G_Elemene | A_bisabolol, |
| | | | | B_curcumene, |
| | | | | Humulene |
| 27 | Xylans | Total Terpenes, | | Total Terpenes, |
| | | Linalool, B_farnasene, | | Terpenolene, |
| | | B_Caryophyllene, | | A_bisabolol, |
| | | Humulene | | |

Although DAMPs and PAMPs include a plethora of different compounds belonging to a vast variety of chemical families, including lipids, peptides, and enzymes, the broad majority of PAMPs and DAMPs that have been characterized to date are polysaccharides. These may be homopolysaccharides (i.e., homoglycans) composed of a single sugar (e.g., chitin, made exclusively from N-acetylglucosamine; and polygalactaronic acid, a degradation product of esterified of pectin), a repeating heterosugar (e.g., hyaluronic acid, a polymer of disaccharides composed of D-glucuronic acid and N-acetyl-D-glucosamine, linked via alternating β-(1-→4) and β-(1-→3) glycosidic bonds), or heteropolysaccharides containing several monomer constituents such as the complex bacterial exopolysaccharides and plant-derived pectin component of hemicellulose. The identity of the monomers, as well as their abundance, frequency, and order, as well as the chemical bond positions which connect the monomers ultimately determine the identity of a specific polysaccharide.

While many scientific publications have demonstrated the effects of these compounds as conjugates of lipids (i.e., lipopolysaccharides) or peptides (i.e., peptidoglycans), topical exposure of plants to such compounds, whether from bacteria, fungus, arthropod, or plant origin, is expected to induce similar plant response pathways owing to some combination of epitopes being recognized by the plant receptors. Pectins are acidic polysaccharides ranging in complexity from simple oligogalacturonans composed of linear chains of free and methyl-esterified galacturonic acid, so-called homogalacturonan (HG), to the more complex rhamnogalacturonans I (RGI) and II (RGII), with six and 11 different glycosyl monomer units, respectively (Gallego-Giraldo et al., 2018).

Enzymes are also important elicitors of plant-defense response, since in most plant pathogens, the initial elicitor and virulence determinant produced may be cell wall-degrading enzymes that facilitate infiltration. Elicitor enzymes include pectinases (e.g., pectolyase, pectozyme, and polygalacturonase), hemicellulases (e.g., Endoxylanase and β-D xylosidase), and lignolytic enzymes such as Ligninolytic enzymes Laccase; Lignin peroxidase; Manganese peroxidase; Versatile peroxidase, Cellulose degrading enzymes (cellulases) such as β-glucosidase, also named Cellobiase are also elicitors due to their catalysis of elicitor molecules such as cellobiose (He et al., 2023). Counterintuitively, application of cell-wall-degrading enzymes to growing plants can be beneficial to plant health due to their defense-response eliciting effect. Endogenous plant enzymes, such as chitinase and β-1,3 glucanases, which are upregulated in response to such pathogen attacks, are also beneficial elicitors which improve plant resistance to pathogens (dos Santos et. al 2023).

These compounds are likewise proposed to induce defense pathways as damage-signaling molecules. Additional DAMP elicitors may be the degradation products or derivatives of plant constituent lignin. Lignin is a complex polyphenol biopolymer primarily present in plant secondary cell walls of the plant vasculature, together with pectins, hemicelluloses, cellulose, and structural proteins. Some research on cannabis has reported limited or no effect of wounding on cannabinoid content of C. sativa (Park et. al, 2009), suggesting DAMPs may play a minor role in cannabinoid synthesis.

However, considering other reports that have found the monomeric composition of lignin-derived DAMPs (e.g., Sinapyl alcohol, Coniferyl alcohol, or 4-Coumaryl alcohol) is the key determinant of which defense pathway is activated in an affected plant (Gallego-Giraldo et al., 2018), and the variability between plant cultivars, variable effects of different lignin (whether it be plant origin or purification method) is plausible. Here, application of lignins from different plant origins (e.g., pine, hardwood, and grass), containing different quantities and ratios of monomer units of Sinapyl alcohol, Coniferyl alcohol, or 4-Coumaryl alcohol, showed considerable positive impact on plant yield and secondary metabolite profile.

Likewise, all of the elicitors tested, whether polysaccharidic, polyphenolic, or enzymatic, and from various biological origins (plants, bacteria, arthropods, fungi) had a biological effect on at least one cultivar, whether protecting against mold (as observed in FIGS. 1A-C and Table 1), increasing yield (as observed in Table 2), increasing total cannabinoid or terpenoid content (as observed in Tables 3A and 3B), or concentration of a specific cannabinoid or terpenoid.

Such effects may be cryptically related, since lowering the pathogen load in asymptomatic plants would probably impact overall plant physiology and primary production and ultimate yield, while the mechanism of protection would probably also induce a change in the secondary metabolite profile, reflecting the increased production of compounds that have been shown to mitigate pathogen proliferation.

The physicochemical properties of plant biopolymers (e.g., polyphenols and polysaccharides) can vary considerably depending on the preparation methods used and the degree of surface modification. For example, chitosan chemical interactions can be highly variable, depending on its biological origin, deacetylation degree and acetylation pattern, molecular weight, type of chemical modification, pH, concentration, and solubility. This enables tailoring the desired physicochemical properties for use in variable applications in completely different fields.

Pectin, chitosan, and lignin nanoparticles are also used as adjuvants, fillers for encapsulation of hydrophobic/incompatible compounds (e.g., carriers), dispersants, and surfactants due to the various degrees of reactiveness, size, and achievable shapes using different fabrication techniques. Molecular weight, monomer type, and reactivity are highly dependent on the extraction method employed (e.g., acidic environment, alkaline environment, enzymatic environment, and/or extraction temperature) and the source of the biopolymer (e.g., shrimp-derived chitin and fungus-derived chitin).

Embodiments of present invention provide biostimulant compositions including exogenous polysaccharides, peptidoglycans, peptide hydrolyzate, lipopolysaccharides, or enzymes derived from plant, fungal, and/or bacterial sources. These substances act as elicitors or biostimulants, triggering immune responses in the plant.

The biostimulants are applied either as a foliar spray or by substrate-drenching. Application can be performed during all growth phases, from rooting clones to adult plants. In some embodiments of the present invention the composition applied essentially consists of the biostimulant as an elicitor.

The biostimulants significantly affect disease resistance, secondary metabolite production, and total biomass and harvest yield. With regard to effects on disease resistance, the application of these biostimulants enhances the plant's resistance to key fungal pathogens, including Fusarium and Pythium species, as well as Botrytis cinerea and Golovinomyces spp. This increased resistance leads to reduced incidence of diseases such as vascular wilt, seedling damping-off, crown rot, gray mold, and powdery mildew (PM).

With regard to effects on secondary metabolite production, regular application of the biostimulants significantly increases the concentration of cannabinoids and terpenoids in Cannabis sativa. This includes an increase in major cannabinoids such as cannabidiolic acid (CBDA) and Δ9-tetrahydrocannabinolic acid (THCA).

With regard to effects on total biomass and harvest yield, regular application of the biostimulants significantly increases the total biomass of treated plants, including inflorescence total dry weight. Enhanced disease resistance leads to lower crop losses and higher yield. Increased secondary metabolite production enhances the medicinal value of Cannabis sativa, particularly in terms of cannabinoid and terpenoid profiles.

While the present invention has been described with respect to a limited number of embodiments, it will be appreciated that many variations, modifications, equivalent structural elements, combinations, sub-combinations, and other applications of the present invention may be made.

### INCORPORATION BY REFERENCE

All references, articles, publications, patents, patent publications, and patent applications cited herein and in the Literature section below are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### LITERATURE

Sirangelo T M, Ludlow R A, Spadafora N D. Molecular Mechanisms Underlying Potential Pathogen Resistance in Cannabis sativa. Plants 2023, 12, 2764.
Punja Z K. Emerging diseases of Cannabis sativa and sustainable management. Pest Manag. Sci. 2021;77: 3857-3870.
Kongala S I, Kondreddy A. A review on plant and pathogen derived carbohydrates, oligosaccharides and their role in plant's immunity. Carbohydrate Polymer Technologies and Applications 6 (2023) 100330.
Garrido et al. Exogenous application of stress-related signaling molecules affect growth and cannabinoid accumulation in medical cannabis (Cannabis sativa L.). Plant Sci., 20 Dec. 2022. Volume 13, 2022.
Mirzamohammad E, Alirezalu A, Alirezalu K, Norozi A, Ansari A. Improvement of the antioxidant activity, phytochemicals, and cannabinoid compounds of Cannabis sativa by salicylic acid elicitor. Food Sci Nutr. 2021; 9:6873-6881.
Aizpurua-Olaizola O, Soydaner U, Öztürk E, Schibano D, Yilmaz Simsir Y, Patricia Navarro P, Nestor Etxebarria N, Usobiaga A. Evolution of the Cannabinoid and Terpene Content during the Growth of Cannabis sativa Plants from Different Chemotypes. J. Nat. Prod. 2016, 79, 324-331.
Jin, D., Dai, K., Xie, Z. et al. Secondary Metabolites Profiled in Cannabis Inflorescences, Leaves, Stem Barks, and Roots for Medicinal Purposes. Sci Rep 10, 3309 (2020).
Welling M T, Deseo M A, Bacic A, Doblin M S. Biosynthetic origins of unusual cannabimimetic phytocannabinoids in Cannabis sativa L: A review. Phytochemistry Volume 201, September 2022, 113282.
Rouphael Y, Colla G. Editorial: Biostimulants in Agriculture. Front. Plant Sci. 11:40 (2020).
Park, S H., Pauli, C. S., Gostin, E. L. et al. Effects of short-term environmental stresses on the onset of cannabinoid production in young immature flowers of industrial hemp (Cannabis sativa L.). J Cannabis Res 4, 1 (2022).
Yee-Shan Ku Y S, Sintaha M, Cheung M Y, Lam H M. Plant Hormone Signaling Crosstalks between Biotic and Abiotic Stress Responses. Int. J. Mol. Sci. 2018, 19, 3206.
Soohyun Oh, Doil Choi; Receptor-mediated nonhost resistance in plants. Essays Biochem 30 Sep. 2022; 66 (5): 435-445.
Punja Z K, Collyer D, Scott C, Lung S, Holmes J and Sutton D (2019) Pathogens and Molds Affecting Production and Quality of Cannabis sativa L. Front. Plant Sci. 10:1120.
Belthazar C. et. Al. Expression of Putative Defense Responses in Cannabis Primed by Pseudomonas and/or Bacillus Strains and Infected by Botrytis cinerea. Front. Plant Sci., 25 Nov. 2020 Sec. Plant Pathogen Interactions. Volume 11-2020.
Koltai H, Namdar D. Cannabis Phytomolecule ‘Entourage’: From Domestication to Medical Use. Trends Plant Sci. 2020 October;25(10):976-984.
Lei et al. Biotechnol Biofuels (2021) 14:147.
Matthias Wü. Smell of Stress: Identification of Induced Biochemical Pathways Affecting the Volatile Composition and Flavor Quality of Crops.
Chacon, F. T .; Raup-Konsavage, W. M .; Vrana, K. E.; Kellogg, J. J. Secondary Terpenes in Cannabis sativa L .: Synthesis and Synergy. Biomedicines 2022, 10, 3142.
Sands L B, Haiden S R, Ma Y, Berkowitz G A. Hormonal control of promoter activities of Cannabis sativa prenyltransferase 1 and 4 and salicylic acid mediated regulation of cannabinoid biosynthesis. Scientific Reports | (2023) 13:8620.
Jalali S, Salami S A, Sharifi M, Sohrabi S. Signaling compounds elicit expression of key genes in cannabinoid pathway and related metabolites in cannabis. Industrial Crops and Products Volume 133, July 2019, Pages 105-110.
Jamiołkowska A., Natural Compounds as Elicitors of Plant Resistance Against Diseases and New Biocontrol Strategies, Agronomy 2020, 10, 173.
Stasinska-Jakubas M, Hawrylak-Nowak B. Protective, Biostimulating, and Eliciting Effects of Chitosan and Its Derivatives on Crop Plants. Molecules 2022, 27, 2801.
Gallego-Giraldo et. al. Elicitors and defense gene induction in plants with altered lignin Compositions. New Phytologist (2018) 219: 1235-1251.
He J, Kong M, Qian Y, Gong M, Lv G, Song J. Cellobiose elicits immunity in lettuce conferring resistance to Botrytis cinerea. J Exp Bot. 2023 Feb. 5;74(3): 1022-1038.
Kostanda E, Khatib S. Biotic stress caused by Tetranychus urticae mites elevates the quantity of secondary metabolites, cannabinoids and terpenes, in Cannabis sativa L., Industrial Crops and Products, Volume 176, 2022, 114331, ISSN 0926-6690.
dos Santos, C .; Franco, O. L. Pathogenesis-Related Proteins (PRs) with Enzyme Activity Activating Plant Defense Responses. Plants 2023, 12, 2226.
Kongala S I and Kondreddy A. (2023) A review on plant and pathogen derived carbohydrates, oligosaccharides and their role in plant's immunity. Carbohydrate Polymer Technologies and Applications, Volume 6,2023, 100330,ISSN 2666-8939
Anil, K, Das, S N, & Podile, A R (2014). Induced defense in plants: A short overview. Proceedings of the National Academy of Sciences India Section B-Biological Sciences, 84(3), 669-679.
Wiesel, L, Newton, A C, Elliott, I, Booty, D, Gilroy, E M, Birch, P R J, & Hein, I (2014). Molecular effects of resistance elicitors from biological origin and their potential for crop protection. Frontiers in Plant Science, 5.
Zipfel, C (2014). Plant pattern-recognition receptors. Trends in Immunology, 35(7), 345-351.
Cui, H, Tsuda, K, & Parker, J E (2015). Effector-triggered immunity: From pathogen perception to robust defense. Annual Review of Plant Biology, 66.
Chianese G, Lopatriello A, Schiano-Moriello A, Caprioglio D, Mattoteia D, Benetti E, Ciceri D, Arnoldi L, De Combarieu E, Vitale R M, Amodeo P, Appendino G, De Petrocellis L, Taglialatela-Scafati O. Cannabitwinol, a Dimeric Phytocannabinoid from Hemp, Cannabis sativa L., Is a Selective Thermo-TRP Modulator. J Nat Prod. 2020 Sep. 25;83(9):2727-2736.
Citti, C., Linciano, P., Russo, F. et al. A novel phytocannabinoid isolated from Cannabis sativa L. with an in vivo cannabimimetic activity higher than Δ9-tetrahydrocannabinol: Δ9-Tetrahydrocannabiphorol. Sci Rep 9, 20335 (2019).
Huizenga M N, Sepulveda-Rodriguez A, Forcelli P A. Preclinical safety and efficacy of cannabidivarin for early life seizures. Neuropharmacology. 2019 April; 148:189-198.
Jadoon K A, Ratcliffe S H, Barrett D A, Thomas E L, Stott C, Bell J D, O′Sullivan SE, Tan G D. Efficacy and Safety of Cannabidiol and Tetrahydrocannabivarin on Glycemic and Lipid Parameters in Patients With Type 2 Diabetes: A Randomized, Double-Blind, Placebo-Controlled, Parallel Group Pilot Study. Diabetes Care. 2016 October; 39(10): 1777-86.
du Jardin, Patrick. (2012). The science of plant biostimulants-a bibliographic analysis. Final Report. 27-30.
Draft Guidance for Plant Regulator Label Claims, Including Plant Biostimulant, United States Environmental Protection Agency (US-EPA))

What is claimed is:

1. A method for improving desired qualities in Cannabis sativa L., the method comprising the step of:
(a) applying a composition having at least one biostimulant, as an elicitor, to Cannabis sativa L., wherein said at least one biostimulant is at least one material selected from the group consisting of: an exogenous polysaccharide, a peptidoglycan, a peptide, a lipopolysaccharide, a modified lipid, and an enzyme, wherein said at least one biostimulant is derived from at least one source material selected from the group consisting of: plants, bacteria, archaebacteria, fungi, animals, protozoa, algae, and viruses, and wherein said at least one biostimulant is known to be sensed by plant surface-receptors to elicit a plant defense-response.

2. The method of claim 1, wherein said at least one biostimulant is at least one material selected from the group consisting of: a Pathogen-Associated Molecular Pattern (PAMP), a Microbial-Associated Molecular Pattern (MAMP), and an Herbivore-Associated Molecular Patterns (HAMP), and wherein said PAMP and said HAMP are derived from at least one source material selected from the group consisting of: fungi, bacteria, archaebacteria, lichen, mollusks, insects, arthropods, oomycete, viruses, algae, and protozoa.

3. The method of claim 1, wherein said enzyme is a cell-wall-degrading enzyme.

4. The method of claim 3, wherein said cell-wall-degrading enzyme is at least one material selected from the group consisting of: ligninolytic enzymes, hemicellulases, pectinases, and cellulases.

5. The method of claim 1, wherein at least one of said at least one biostimulant is a Damage-Associated Molecular Pattern (DAMP), and wherein said DAMP is derived from at least one source material selected from the group consisting of: plants, moss, lichen, algae, enzymes, biopolymers, and degradation products thereof.

6. The method of claim 5, wherein said biopolymers are materials selected from the group consisting of: cellulose, hemicellulose, pectin, and degradation products thereof.

7. The method of claim 5, wherein said biopolymers are materials selected from the group consisting of: lignin polyphenols and degradation products thereof.

8. The method of claim 5, wherein said enzymes are Pathogenesis-Related Proteins (PRs).

9. The method of claim 8, wherein said Pathogenesis-Related Proteins are materials selected from the group consisting of: ß-1,3 glucanases and chitinases.

10. The method of claim 1, wherein said plant defense-response is an increase in at least one aspect selected from the group consisting of: a disease resistance, a secondary metabolite, and a biomass yield.

11. The method of claim 10, wherein said disease resistance includes resistance to important plant fungal pathogens in all phases of growth for both rooting clones and adult cannabis sativa plants.

12. The method of claim 10, wherein said secondary metabolite is at least one material selected from the group consisting of: a cannabinoid and a terpenoid, and wherein said increase in said secondary metabolite is in a cultivar-dependent manner.

13. The method of claim 1, wherein said step of applying said composition is performed by using at least one technique selected from the group consisting of: employing a foliar spray and employing substrate-drenching.

14. The method of claim 1, wherein said at least one biostimulant is in at least one form selected from the group consisting of: a water-soluble material, a crystalline material, an amorphous material, a non-soluble material, a suspended colloidal particle, a nanoparticle constituent, a phase-boundary aggregate, a micellar suspension, and a precipitate.

15. The method of claim 1, wherein said exogenous polysaccharide includes at least one material selected from the group consisting of: a polysaccharide conjugate, a polysaccharide derivative, a surface-modified polysaccharide, a depolymerized polysaccharide, and an oligomer-fragmented polysaccharide.
